(19) United States
(12) Patent Application Publication
(10) Pub. No.: US 2026/0209155 A1
(43) Pub. Date: Jul. 23, 2026

(54) METHOD FOR REGENERATING BLENDED FIBER

(71) Applicant: HONDA MOTOR CO., LTD., Tokyo (JP)

(72) Inventors: Akihiro OGUMA, Saitama (JP); Ryo NAKAI, Saitama (JP)

(21) Appl. No.: 19/411,372

(22) Filed: Dec. 8, 2025

(30) Foreign Application Priority Data
Jan. 22, 2025 (JP) 2025-008978

(51) Int. Cl. C07C 51/09; C07C 51/43; C08J 11/24

(52) U.S. Cl. CPC C07C 51/09; C07C 51/43; C08J 11/24

(57) ABSTRACT

Provided is a method for regenerating a blended fiber. The method includes depolymerizing a blended fiber including polyester fiber and wool in a solution in which a strong base is dissolved in a solvent to obtain a first reaction solution. The solvent includes tetrahydrofuran and methanol. The ratio of methanol to the total volume of tetrahydrofuran and methanol is 20% by volume or more and 40% by volume or less.

This application is based on and claims the benefit of priority from Japanese Patent Application No. 2025-008978, filed on 22 Jan. 2025, the content of which is incorporated herein by reference.

### BACKGROUND OF THE INVENTION

### Field of the Invention

The present invention relates to a method for regenerating a blended fiber including polyester fiber and wool.

### Related Art

In recent years, efforts have been made to significantly reduce waste generation through waste prevention, reduction, recycling, and reuse. In order to achieve this, research and development relating to the recycling of blended fibers has been carried out.

Japanese Unexamined Patent Application, Publication No. 2024-125271 discloses a method for recycling waste blend textiles including polyester fibers and cotton staple fibers. At this time, the polyester fibers are depolymerized in a basic aqueous solution under a controlled environment to obtain treated textiles including cotton staple fibers.
Patent Document 1: Japanese Unexamined Patent Application, Publication No. 2024-125271

### SUMMARY OF THE INVENTION

However, in the method disclosed in Japanese Unexamined Patent Application, Publication No. 2024-125271, the recovery rate of terephthalic acid, which is a constituent unit of polyester fiber, cannot be improved when regenerating a blended fiber including polyester fiber and wool.

An object of the present invention is to provide a method for regenerating a blended fiber capable of improving the recovery rate of terephthalic acid.

A first aspect of the present invention is a method for regenerating a blended fiber. The method includes depolymerizing a blended fiber including polyester fiber and wool in a solution in which a strong base is dissolved in a solvent to obtain a first reaction solution. The solvent includes tetrahydrofuran and methanol. A ratio of the methanol to a total volume of the tetrahydrofuran and the methanol is 20% by volume or more and 40% by volume or less.

A second aspect of the present invention is a method for regenerating a blended fiber. The method includes:
depolymerizing a blended fiber including polyester fiber and wool in an aqueous solution of a strong base to obtain a second reaction solution; filtering the second reaction solution to obtain a filtered material; and depolymerizing the filtered material in a solution in which a strong base is dissolved in a solvent to obtain a first reaction solution. The solvent includes tetrahydrofuran and methanol. A ratio of the methanol to a total volume of the tetrahydrofuran and the methanol is 20% by volume or more and 40% by volume or less.

In a third aspect of the present invention according to the first or second aspect, the method further includes distilling off the solvent from the first reaction solution to obtain a solid; adding water and an organic solvent to the solid and filtering to obtain a filtrate; separating the filtrate to obtain an aqueous layer; and adding a strong acid to the aqueous layer to form a precipitate.

In a fourth aspect of the present invention according to the third aspect, the blended fiber further includes polyamide fiber. The method further includes immersing in methanol the precipitate formed by adding the strong acid to the aqueous layer.

According to the present invention, it is possible to provide a method for regenerating a blended fiber capable of improving the recovery rate of terephthalic acid.

### BRIEF DESCRIPTION OF THE DRAWINGS

FIG. 1 is an infrared transmission spectrum of a precipitate of Example 1; and

FIG. 2 is an infrared transmission spectrum of a precipitate of Example 2.

### DETAILED DESCRIPTION OF THE INVENTION

Hereinafter, embodiments of the present invention will be described.

### First Aspect of Method for Regenerating Blended Fiber

The method for regenerating a blended fiber of the present embodiment includes a step of depolymerizing a blended fiber including polyester fiber and wool in a solution in which a strong base is dissolved in a solvent to obtain a first reaction solution. Here, the solvent includes tetrahydrofuran and methanol. The ratio of methanol to the total volume of tetrahydrofuran and methanol is 20% by volume or more and 40% by volume or less, and preferably 25% by volume or more and 40% by volume or less. Since the ratio of methanol to the total volume of tetrahydrofuran and methanol is 20% by volume or more, depolymerization of the wool is suppressed, and since the ratio is 40% by volume or less, depolymerization of the polyester fiber is accelerated and depolymerization of the wool is suppressed. As a result, the recovery rate of terephthalic acid, which is a constituent unit of the polyester fiber, is improved.

The strong base is not particularly limited as long as it can depolymerize the polyester fiber, and examples thereof include sodium hydroxide, potassium hydroxide, barium hydroxide, strontium hydroxide, and lithium hydroxide.

The depolymerization temperature is not particularly limited, but is, for example, 20° C. or more and 30° C. or less.

Water may be added after the depolymerization of the blended fiber.

The method for regenerating a blended fiber of the present embodiment may further include a step of distilling off the solvent from the first reaction solution to obtain a solid, a step of adding water and an organic solvent to the solid and filtering to obtain a filtrate, a step of separating the filtrate to obtain an aqueous layer, and a step of adding a strong acid to the aqueous layer to form a precipitate.

The organic solvent is not particularly limited as long as it can purify terephthalate as a depolymerization product, and examples thereof include ethyl acetate.

The strong acid is not particularly limited as long as it can precipitate terephthalic acid, and examples thereof include sulfuric acid.

The blended fiber may further include fibers other than polyester fiber and wool. The fibers other than polyester fiber and wool are not particularly limited, and examples thereof include cotton, acrylic fiber, polyamide fiber, urethane fiber, and rayon.

When the blend fiber includes polyamide fiber, the method for regenerating a blended fiber of the present embodiment preferably further includes a step of immersing in methanol the precipitate formed by adding the strong acid to the aqueous layer. As a result, the polyamide fiber contained in the precipitate is dissolved in methanol and removed.

### Second Aspect of Method for Regenerating Blended Fiber

The method for regenerating a blended fiber of the present embodiment includes a step of depolymerizing a blended fiber including polyester fiber and wool in an aqueous solution of a strong base to obtain a second reaction solution, and a step of filtering the second reaction solution to obtain a filtered material. This removes the wool from the blended fiber.

The strong base is not particularly limited as long as it can depolymerize wool, and examples thereof include sodium hydroxide, potassium hydroxide, barium hydroxide, strontium hydroxide, and lithium hydroxide.

The depolymerization temperature is not particularly limited, but is, for example, 20° C. or higher and 65° C. or less.

The method for regenerating a blended fiber of the present embodiment further includes a step of depolymerizing the filtered material in a solution in which a strong base is dissolved in a solvent to obtain a first reaction solution. Here, the solvent includes tetrahydrofuran and methanol. The ratio of methanol to the total volume of tetrahydrofuran and methanol is 20% by volume or more and 40% by volume or less, and preferably 25% by volume or more and 40% by volume or less. Since the ratio of methanol to the total volume of tetrahydrofuran and methanol is 20% by volume or more, depolymerization of wool is suppressed, and since the ratio is 40% by volume or less, depolymerization of the polyester fiber is accelerated and depolymerization of the wool is suppressed. As a result, the recovery rate of terephthalic acid, which is a constituent unit of the polyester fiber, is improved.

The strong base is not particularly limited as long as it can depolymerize the polyester fiber, and examples thereof include sodium hydroxide, potassium hydroxide, barium hydroxide, strontium hydroxide, and lithium hydroxide.

The depolymerization temperature is not particularly limited, but is, for example, 20° C. or higher and 30° C. or less.

Water may be added after the depolymerization of the blended fiber.

The method for regenerating a blended fiber of the present embodiment may further include a step of distilling off the solvent from the first reaction solution to obtain a solid, a step of adding water and an organic solvent to the solid and filtering to obtain a filtrate, a step of separating the filtrate to obtain an aqueous layer, and a step of adding a strong acid to the aqueous layer to form a precipitate.

The organic solvent is not particularly limited as long as it can purify terephthalate as a depolymerization product, and examples thereof include ethyl acetate.

The strong acid is not particularly limited as long as it can precipitate terephthalic acid, and examples thereof include sulfuric acid.

The blended fiber may further include fibers other than polyester fiber and wool. The fibers other than polyester fiber and wool are not particularly limited, and examples thereof include cotton, acrylic fiber, polyamide fiber, urethane fiber, and rayon.

When the blend fiber includes polyamide fiber, the method for regenerating a blended fiber of the present embodiment preferably further includes a step of immersing in methanol the precipitate formed by adding the strong acid to the aqueous layer. As a result, the polyamide fiber contained in the precipitate is dissolved in methanol and removed.

Although the embodiments of the present invention have been described above, the present invention is not limited to the above-described embodiments, and the above-described embodiments may be modified as appropriate within the scope of the gist of the present invention.

### EXAMPLES

Examples of the present invention will be described below, but the present invention is not limited to these examples.

### Infrared Transmission Spectrum

The infrared transmission spectrum of a precipitate was measured using an infrared spectrophotometer (IR).

### Example 1

### Blended Fiber

3.99 g of blended fiber consisting of 1.89 g of cotton, 1.24 g of polyester fiber, 0.34 g of acrylic fiber, 0.30 g of wool, 0.07 g of nylon as polyamide fiber, 0.04 g of urethane fiber, and 0.11 g of rayon was regenerated. Here, the polyester fiber consists of a terephthalic acid unit and an ethylene glycol unit.

### Depolymerization

3.99 g of the blended fiber was reacted at 60° C. for 5 hours under reflux in a solution of 2 g of sodium hydroxide dissolved in 100 mL of water, and then the mixture was filtered under reduced pressure to obtain 3.70 g of a filtrate and a filtered material. Next, 30 mL of a 1 mol/L sulfuric acid aqueous solution was added to the filtrate to form a precipitate, which was then filtered under reduced pressure and dried overnight at 80° C. to obtain 0.05 g of a precipitate.

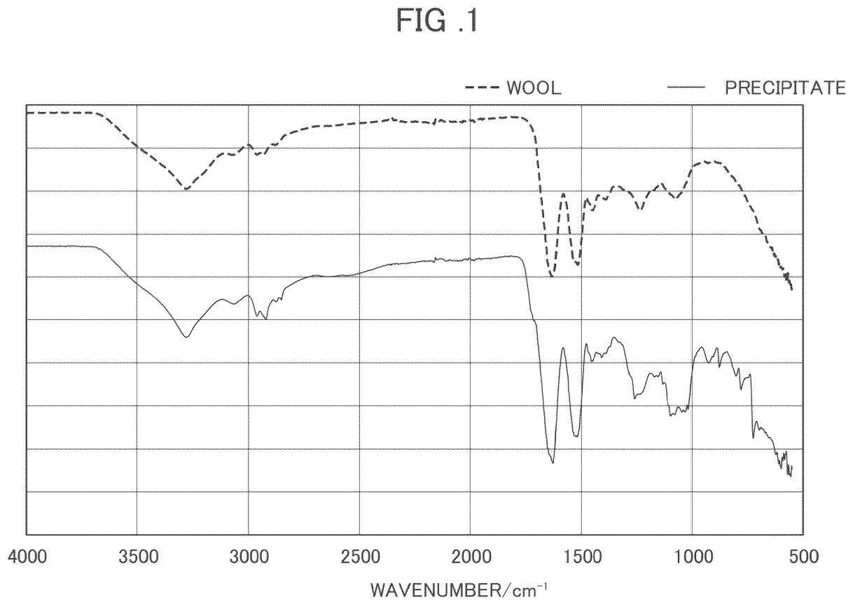

FIG. 1 shows an infrared transmission spectrum of the precipitate. FIG. 1 also shows an infrared transmission spectrum of wool.

It can be seen from FIG. 1 that the precipitate is a depolymerized product of wool substantially free of polyester.

### Depolymerization

3.70 g of the filtered material was reacted at room temperature for 5 hours in a solution prepared by dissolving 2 g of sodium hydroxide in a mixed solvent of 25 mL of methanol and 75 mL of tetrahydrofuran, and then 100 mL of water was added and the mixture was stirred overnight to obtain a reaction solution.

### Solvent Distillation

The solvent was distilled off from the reaction solution while heating at 45° C. using an evaporator to obtain a solid.

### Extraction

150 mL of water and 150 mL of ethyl acetate were added to the solid and the mixture was stirred and then filtered under reduced pressure to obtain 2.50 g of a filtrate and a filtered material. Next, the filtrate was separated using a separatory funnel, and then the ethyl acetate layer was removed to obtain an aqueous layer. Next, 150 mL of ethyl acetate was added to the aqueous layer, and the mixture was separated using a separatory funnel, and the ethyl acetate layer was removed. This procedure was repeated twice to obtain an aqueous layer. Next, 30 mL of a 1 mol/L sulfuric acid aqueous solution was added to the aqueous layer to form a precipitate, which was then filtered under reduced pressure to obtain a filtered material.

### Post-Treatment

The filtered material was immersed in 70 mL of methanol for 30 minutes and then filtered under reduced pressure to obtain 1.01 g of a filtrate and a filtered material. At this time, it was identified by liquid chromatography mass spectrometry (LC-MS) that the filtered material was terephthalic acid, and the recovery rate of terephthalic acid was 94%.

### Example 2

### Blended Fiber

4.03 g of a blended fiber of polyester fiber and wool (mass ratio 7:3) was regenerated as the blended fiber. Here, the polyester fiber consists of a terephthalic acid unit and an ethylene glycol unit.

### Depolymerization

4.03 g of the blended fiber was reacted at 60° C. for 5 hours under reflux in a solution of 2 g of sodium hydroxide dissolved in 100 mL of water, and then the mixture was filtered under reduced pressure to obtain 2.99 g of a filtrate and a filtered material. Next, 30 mL of a 1 mol/L sulfuric acid aqueous solution was added to the filtrate to form a precipitate, which was then filtered under reduced pressure and dried overnight at 80° C. to obtain 0.07 g of a precipitate.

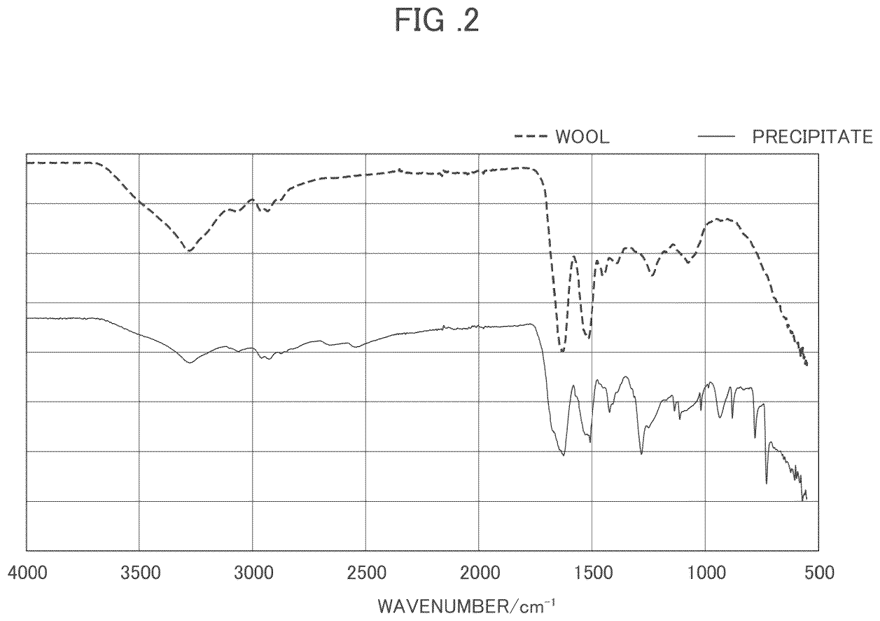

FIG. 2 shows an infrared transmission spectrum of the precipitate. FIG. 2 also shows an infrared transmission spectrum of wool.

It can be seen from FIG. 2 that the precipitate is a depolymerized product of wool substantially free of polyester.

### Depolymerization

2.99 g of the filtered material was reacted at room temperature for 5 hours in a solution prepared by dissolving 2 g of sodium hydroxide in a mixed solvent of 25 mL of methanol and 75 mL of tetrahydrofuran, and then 100 mL of water was added and the mixture was stirred overnight to obtain a reaction solution.

### Solvent Distillation

The solvent was distilled off from the reaction solution while heating at 45° C. using an evaporator to obtain a solid.

### Extraction

150 mL of water and 150 mL of ethyl acetate were added to the solid, and the mixture was stirred and then filtered under reduced pressure to obtain 0.95 g of a filtrate and a filtered material. Next, the filtrate was separated using a separatory funnel, and then the ethyl acetate layer was removed to obtain an aqueous layer. Next, 150 mL of ethyl acetate was added to the aqueous layer, and the mixture was separated using a separatory funnel, and the ethyl acetate layer was removed. This procedure was repeated twice to obtain an aqueous layer. Next, 30 mL of a 1 mol/L sulfuric acid aqueous solution was added to the aqueous layer to form a precipitate, which was then filtered under reduced pressure to obtain 2.44 g of a filtered material. At this time, it was identified by liquid chromatography mass spectrometry (LC-MS) that the filtered material was terephthalic acid, and the recovery rate of terephthalic acid was 100%.

### Example 3

### Blended Fiber

4.00 g of a blended fiber of polyester fiber and wool (mass ratio 7:3) was regenerated as the blended fiber. Here, the polyester fiber consists of a terephthalic acid unit and an ethylene glycol unit.

### Depolymerization

4.00 g of the blended fiber was reacted at room temperature for 5 hours in a solution prepared by dissolving 2 g of sodium hydroxide in a mixed solvent of 25 mL of methanol and 75 mL of tetrahydrofuran, and then 150 mL of water was added and the mixture was stirred overnight to obtain a reaction solution.

### Solvent Distillation

The solvent was distilled off from the reaction solution while heating at 45° C. using an evaporator to obtain a solid.

### Extraction

150 mL of water and 150 mL of ethyl acetate were added to the solid and the mixture was stirred, and then filtered under reduced pressure to obtain 1.05 g of a filtrate and a filtered material. Next, the filtrate was separated using a separatory funnel, and then the ethyl acetate layer was removed to obtain an aqueous layer. Next, 150 mL of ethyl acetate was added to the aqueous layer, and the mixture was separated using a separatory funnel, and the ethyl acetate layer was removed. This procedure was repeated twice to obtain an aqueous layer. Next, 30 mL of a 1 mol/L sulfuric acid aqueous solution was added to the aqueous layer to form a precipitate, which was then filtered under reduced pressure to obtain 2.60 g of a filtered material. At this time, it was identified by liquid chromatography mass spectrometry (LC-MS) that the filtered material was terephthalic acid, and the recovery rate of terephthalic acid was 100%.

What is claimed is:

1. A method for regenerating a blended fiber, the method comprising:
depolymerizing a blended fiber comprising polyester fiber and wool in a solution in which a strong base is dissolved in a solvent to obtain a first reaction solution,
wherein the solvent comprises tetrahydrofuran and methanol, and a ratio of the methanol to a total volume of the tetrahydrofuran and the methanol is 20% by volume or more and 40% by volume or less.

2. A method for regenerating a blended fiber, the method comprising:
depolymerizing a blended fiber comprising polyester fiber and wool in an aqueous solution of a strong base to obtain a second reaction solution;
filtering the second reaction solution to obtain a filtered material; and
depolymerizing the filtered material in a solution in which a strong base is dissolved in a solvent to obtain a first reaction solution,
wherein the solvent comprises tetrahydrofuran and methanol, and a ratio of the methanol to a total volume of the tetrahydrofuran and the methanol is 20% by volume or more and 40% by volume or less.

3. The method for regenerating a blended fiber according to claim 1, the method further comprising:
distilling off the solvent from the first reaction solution to obtain a solid;
adding water and an organic solvent to the solid and filtering to obtain a filtrate;
separating the filtrate to obtain an aqueous layer; and
adding a strong acid to the aqueous layer to form a precipitate.

4. The method for regenerating a blended fiber according to claim 3,
wherein the blended fiber further comprises polyamide fiber, and
wherein the method further comprises immersing in methanol the precipitate formed by adding the strong acid to the aqueous layer.
